(19) United States
(12) Patent Application Publication
(10) Pub. No.: US 2013/0260364 A1
(43) Pub. Date: Oct. 3, 2013

(54) Multifunctional Bioreactor system and methods for cell sorting and culturing

(71) Applicant: Yongxin Zhang, Carrollton, TX (US)

(72) Inventor: Yongxin Zhang, Carrollton, TX (US)

(21) Appl. No.: 13/435,250

(22) Filed: Mar. 30, 2012

(51) Int. Cl. C12Q 3/00; G01N 33/53; C12Q 1/04; C12M 1/42; C12M 3/00

(52) U.S. Cl. 435/3; 435/287.1; 435/286.1; 435/34; 435/7.1

(57) ABSTRACT

The invention relates to a multifunctional bioreactor for cell sorting and cell culture in vitro. Said bioreactor comprises five main elements, including an adjustable magnetic field, a multifunctional cell supporting system, a protective perfusion system and a computerized control system. Said methods are for the application of the said bioreactor. The said bioreactor has the functions of cell expansion, cell directed differentiation and cell separation (sorting). It allows its all functions carried out in one reaction chamber.

### CROSS-REFERENCES TO RELATED APPLICATION

### US Patents:

| | | |
|---|---|---|
| 5,968,820 | October 1999 | Zborowski |
| 6,120,735 | September 2000 | Zborowski |
| 7,339,045 | January 2009 | Oakey |
| 7,339,045 | March 2008 | Rothschild |
| 8,071,395 | December 2011 | Davis |
| 8,123,199 | February 2012 | Terentiev |
| 1,269,189 | June 1918 | Kadish |
| 1,505,204 | August 1924 | Kiernan |
| 2,793,166 | May 1957 | Alden |
| 3,002,895 | October 1961 | Freedman |
| 3,647,397 | March 1972 | Coleman |
| 3,900,186 | August 1975 | Balas |
| 3,962,892 | June 1976 | Garlinghouse |
| 4,162,855 | July 1979 | Bender |
| 4,209,259 | June 1980 | Rains et al. |
| 4,356,967 | November 1982 | Lunick |
| 4,498,785 | February 1985 | de Bruyne |
| 4,668,632 | May 1987 | Young et al. |
| 4,711,582 | December 1987 | Kennedy |
| 4,783,172 | November 1988 | Garg |
| 4,808,348 | February 1989 | Rudick et al. |
| 4,870,018 | September 1989 | Lehmann |
| 4,978,616 | December 1990 | Dean, Jr. et al. |
| 5,008,197 | April 1991 | Wergeland et al. |
| 5,061,448 | October 1991 | Mahe et al. |
| 5,205,783 | April 1993 | Dieckert et al. |
| RE34386 | September 1993 | Davidson et al. |
| 5,270,207 | December 1993 | Matsumura et al. |
| 5,401,212 | March 1995 | Marvell et al. |
| 5,501,971 | March 1996 | Freedman et al. |
| 5,591,344 | January 1997 | Kenley et al. |
| 5,656,491 | August 1997 | Cassani et al. |
| 5,727,878 | March 1998 | Sullivan, Jr. |
| 5,750,440 | May 1998 | Vanell et al. |
| 5,779,359 | July 1998 | Gambrill et al. |
| 5,803,137 | September 1998 | Shimotoyodome et al. |
| 5,939,313 | August 1999 | Cheng |
| 5,941,635 | August 1999 | Stewart |
| 5,988,422 | November 1999 | Vallot |
| 6,071,005 | June 2000 | Ekambaram et al. |
| 6,245,555 | June 2001 | Curtis |
| 6,247,840 | June 2001 | Gaffar |
| 6,357,907 | March 2002 | Cleveland et al. |
| 6,379,625 | April 2002 | Zuk, Jr. |
| 6,402,367 | June 2002 | Lu et al. |
| 6,439,756 | August 2002 | Forschner et al. |
| 6,467,946 | October 2002 | Gebrian |
| 6,500,343 | December 2002 | Siddiqi |
| 6,514,137 | February 2003 | Panelli et al. |
| 6,555,011 | April 2003 | Tribelsky et al. |
| 6,637,927 | October 2003 | Lu et al. |
| 6,670,171 | December 2003 | Carll |
| 6,736,906 | May 2004 | Cotte et al. |
| 6,764,859 | July 2004 | Kreuwel et al. |
| 6,923,567 | August 2005 | Bibbo et al. |
| 7,153,021 | December 2006 | Goodwin et al. |
| 7,278,780 | October 2007 | Goodwin et al. |
| 7,384,027 | June 2008 | Terentiev et al. |
| 7,384,783 | June 2008 | Kunas et al. |
| 7,629,167 | December 2009 | Hodge et al. |
| 7,762,716 | July 2010 | Terentiev et al. |
| 2001/0039369 | November 2001 | Terentiev |
| 2002/0082173 | June 2002 | Terentiev |
| 2002/0091371 | July 2002 | Ritter |
| 2002/0105856 | August 2002 | Terentiev |
| 2002/0145940 | October 2002 | Terentiev |
| 2003/0008389 | January 2003 | Carll |
| 2003/0170810 | September 2003 | Vedadi et al. |
| 2003/0226857 | December 2003 | Bibbo et al. |
| 2004/0047232 | March 2004 | Terentiev |
| 2004/0062140 | April 2004 | Cadogan et al. |
| 2004/0218468 | November 2004 | Terentiev |
| 2004/0221897 | November 2004 | Schubmehl et al. |
| 2004/0229335 | November 2004 | Zhang et al. |
| 2004/0252582 | December 2004 | Bucher |
| 2005/0002274 | January 2005 | Terentiev |
| 2005/0117449 | June 2005 | Terentiev |
| 2005/0127215 | June 2005 | Lienhart et al. |
| 2005/0163667 | July 2005 | Krause |
| 2005/0201201 | September 2005 | Terentiev |
| 2005/0239199 | October 2005 | Kunas et al. |
| 2005/0272146 | December 2005 | Hodge et al. |
| 2006/0092761 | May 2006 | Terentiev |
| 2006/0131765 | June 2006 | Terentiev et al. |
| 2006/0270036 | November 2006 | Goodwin et al. |
| 2007/0030759 | February 2007 | Terentiev |
| 2007/0201993 | August 2007 | Terentiev et al. |
| 2007/0220956 | September 2007 | Terentiev |
| 2007/0252290 | November 2007 | Terentiev et al. |
| 2007/0263484 | November 2007 | Terentiev |
| 2008/0008028 | January 2008 | Terentiev et al. |

### Foreign Patent Documents

| | | |
|---|---|---|
| 2,271,583 | April, 2002 | CA |
| 2,017,472 | November, 1971 | DE |
| 3,246,330 | June, 1984 | DE |
| 3,407,370 | August, 1985 | DE |
| 3,818,776 | July, 1989 | DE |
| 19/542,227 | May, 1997 | DE |
| 29/800,818 | March, 1998 | DE |
| 19/705,118 | August, 1998 | DE |
| 19/917,398 | October, 2000 | DE |
| 20/114,076 | October, 2001 | DE |
| 0,033,292 | August, 1981 | EP |
| 0,200,792 | January, 1985 | EP |
| 0,200,792 | November, 1986 | EP |
| 0,343,885 | November, 1989 | EP |
| 0,433,463 | June, 1991 | EP |
| 0,590,473 | April, 1994 | EP |
| 1,462,155 | September, 2004 | EP |
| 2,799,138 | May, 1999 | FR |
| 2,076,677 | December, 1981 | GB |
| 2,202,549 | September, 1988 | GB |
| 61-067476 | April, 1986 | JP |
| 61/212,275 | September, 1986 | JP |
| 631626 | January, 1988 | JP |
| 63-36825 | February, 1988 | JP |
| 03-242297 | October, 1991 | JP |
| 6,153,902 | June, 1994 | JP |
| 9-141079 | December, 1998 | JP |
| 10/313,718 | December, 1998 | JP |
| 10/314,569 | December, 1998 | JP |
| WO9833538 | August, 1998 | WO |
| 9,852,629 | November, 1998 | WO |
| WO0011953 | March, 2000 | WO |
| 03/028,869 | April, 2003 | WO |
| WO 2005/037658 | April, 2005 | WO |
| WO 2005/118771 | December, 2005 | WO |
| WO 2006/002091 | January, 2006 | WO |
| WO 2006/063087 | June, 2006 | WO |
| WO 2007/039600 | April, 2007 | WO |
| WO 2008/040567 | April, 2008 | WO |
| WO 2008/040568 | April, 2008 | WO |

### FIELD OF THE INVENTION

The invention relates to a multifunctional bioreactor for cell culture and cell sorting.

### BACKGROUND OF THE INVENTION

This application relates generally to bioreactors and more particularly bioreactors for growing and separating cells.

Many kinds of cells, especially hematopoietic stem cells and immunocytes, need to be isolated from original samples before they can be efficiently expanded and directed differentiated in culture. This isolation procedure is also called cell sorting or cell separation. Previously, the cell sorting and cell culture have been respectively conducted in separated systems, in which the target cells are isolated first and then are transferred into culture containers. This traditional method is quite cumbersome and has a higher risk for cell contamination and cell loss from the cell sorting to cell culture, in which two completely different devices and systems were involved. Our current invention with a novel design allows these two different procedures completed in one container (chamber), and so minimizes the risk of contamination and target cell loss, and significantly increases the efficiency of the operation.

Some cells are very sensitive to shear-stress in the culture. For example, shear-stress can cause the non-specific differentiation and the increased apoptosis in the stem cell culture, which significantly reduces the efficiency of the stem cell expansion and directed differentiation. The higher shear-stress also causes the more release of non-specific proteins in protein expression, in which the protein of interest takes less proportion in the culture and so result in the increase of protein purification. The static culture has the least shear stress but the cells in static culture normally sit at the bottom of the culture containers, some cells cannot get enough nutrition when cells are at higher density and so not suitable for large scale cell expansion. Some bioreactors, such as NASA's rotation wall vessel (RWV) bioreactor, were designed for reducing the shear-stress. However, these bioreactors have to keep cells in suspension by continuously moving, stirring or/and agitating cells. Once the bioreactor stop running, cells will accumulate somewhere of the bottom but are not evenly distributed, which is harmful for most cell growth. Therefore, though the shear-stress has been reduced in these bioreactors, the reduced shear-stress has to continuously exert on the cultured cells when these bioreactors running. In our current invention, when the bioreactor is at static status, cells are allowed to evenly distribute at the bottom of the culture chamber or on the surface of the magnetic beads. Thus, our invention provides cells the best growth condition in both suspension status and static status.

Some bioreactors use magnet element (specifically blades or vans) controlled by magnet impeller to agitate culture media to keep cells in suspension status. This kind of bioreactor purposely enhances the shear-stress for the culture requirements of a certain cells. In addition to the differences in the application, the bioreactor in our invention does not use blades or canes to be the magnet element, and the magnet beads in our invention actually has no magnetism if they are not in magnet field and they can only gain magnetism when they are placed in magnet field. The magnet beads in our invention are not controlled by impeller but by the changes of magnetic field strength affecting the beads' moving. The proper microenvironments, or so called niches, are very important for the growth of some cells, such as stem cells. Some devices use solid materials to form niches or use gel-like materials to form niches. With these devices, after cells culture, cells need to be rinsed out from niches with a special procedure or the niche-forming materials has to be melted or digested with enzyme to release the target cells. In our current invention, the interspaces among beads naturally from the niches for cell growth, and the cells can be easily released when the beads are lifted by changing the magnet field strength. Some other systems of cell culture attempt to build niches via welled culture plates (such as the common 96-welled plate), micro-chambers (Hung 2005) or micro-sieves (Zhang 2009). The current invented bioreactor is superior to the welled plate because the mobility of the beads creates dynamic microenvironments for cells needing increased flux of medium to grow. It is also superior to micro-chambers and micro-sieves because it poses less of a challenge to manufacture, is easier to sterilize after use, and the size of the niche can be easily modified by adjusting the size of the beads.

Many cell culture containers (chambers) have been designed for bioreactor use, such as common cell culture flasks, gas-permeable bags, rotation wall vessels, and so on. These containers can be used with common perfusion system with which the cells can be diluted and media can be changed. However, during the media change with these containers, the cytokines, proteins and other expensive substances for cell growth and cells' products are removed from the culture simultaneously. And, the efficiency of common dialysis process is not high enough. The cell culture chambers designed in our current invention take the advantage of colloid osmotic force differences between osmosis chambers at two side of culture chamber to allow the media exchange go through the dialysis membranes rapidly without any loss of cytokines, peptides, proteins and other materials in a certain size. This chamber design provides a novel perfusion strategy and the system with it is called as gradient osmosis perfusion system.

Most bioreactors were designed for culturing either adherent or suspension cells. No bioreactors have been reported to support the growth of partial adherent cells yet. The bioreactor in our current invention can be used for the culture of almost any cell, including suspension cells, adherent cells and partial adherent cells.

It is very common to use computer to control bioreactor running, and many bioreactors are programmable. It is emphasized in our current invention that (1) the strength and direction of magnetic fields, (2)the frequencies and speeds of cell culture chamber flip, and (3) frequencies and speeds of the magnet beads as results of above (1) and (2) are controlled by pre-selected programs or/and programs that response to the data it receives from detectors and send feedback to bioreactor.

Similar to the application of computer in bioreactor control, several cell density detectors with some special light sources (such as laser projector) have been designed for monitoring the cell concentration during the cell culture. The data obtained from the detector or sensor is used to determine if the cells need to be diluted if the culture needs media change. In our current invented bioreactor, the cell density detector uses the common light source and the data is specifically used to adjust the strength of the magnetic field, the flipping speed and frequency of cell culture chamber, as well as indirectly adjust the moving speed and frequency of the magnet beads.

### SUMMARY

Bioreactors are typically employed to grow cells within a culture. However, many kinds of cells need to be isolated from original samples before they can be efficiently expanded and directed differentiated in culture. This isolation procedure is also called cell sorting or cell separation. Typically, the cell sorting and cell culture have been conducted in separated systems, in which the target cells are isolated first and then are transferred into culture containers. This traditional method is quite cumbersome and has a higher risk for cell contamination and cell loss from the cell sorting to cell culture, in which two completely different devices and systems are involved.

Additionally, many bioreactors employ rotating impellers or the like for mixing the contents of the bioreactor. Unfortunately, this imparts a shear stress on the cells which may damage the cells, reduces the efficiency of the system, causes a release of waste products, such as non-specific proteins expression, or the like. Additionally, in some bioreactors, cells may aggregate on the bottom of the cell culture chamber or other locations within the cell culture chamber. The aggregation of these cells in the chamber is not conducive to efficient cell growth. Therefore, there exits a significant need for an efficient bioreactor capable of growing and separating cells therewithin while also minimizing shear-stress imparted to the cells.

In one embodiment, a bioreactor system for growing and separating cells comprises a cell culture chamber comprising an interior having a first portion and a second portion; an agitator disposed within the chamber interior, the agitator capable of moving between the interior first portion and interior second portion; and a control system coupled to the cell culture chamber, the control system operable to cause the agitator to move between the interior first portion and interior second portion.

In another embodiment, a method for growing and separating cells comprises providing a chamber comprising an interior having a first portion and a second portion; disposing an agitator within the interior, the agitator capable of moving between the interior first portion and interior second portion; delivering a cell culture media to the chamber interior; transferring target cells to the chamber interior; moving the agitator between the first portion and second portion so as to mix the cells and media; and removing waste from the chamber interior while maintaining a substantial number of the target cells within the chamber interior.

In yet another embodiment, a cell culture chamber interior comprises: a first compartment, the first compartment adapted for fluid communication with a cell culture media reservoir such that the first compartment is capable of receiving cell culture media from the reservoir; a second compartment, wherein the agitator is disposed within the second compartment; a first membrane positioned between the first compartment and second compartment, the first membrane adapted to selectively permit cell culture media to flow from the first compartment to the second compartment; a third compartment, the third compartment adapted for fluid communication with a buffer reservoir such that the third compartment is capable of receiving buffer from the buffer reservoir and wherein the third compartment is further adapted for fluid communication with a waste reservoir such that waste is capable of flowing from the third reservoir to the waste reservoir; and a second membrane positioned between the second compartment and third compartment, the second membrane adapted to selectively permit flow of media and waste from the second compartment to the third compartment.

### BRIEF DESCRIPTION OF THE DRAWINGS

The drawings, when considered in connection with the following description, are presented for the purpose of facilitating an understanding of the subject matter sought to be protected.

FIG. 1 is a schematic view showing an illustrative bioreactor;

FIGS. 2a-2f show a series of schematic views of a bioreactor showing the movement of an agitator within a chamber in series;

FIG. 3 is a schematic isometric view of a chamber;

FIG. 4 is a schematic side-view of a chamber; and

FIG. is a schematic view of an agitator.

### DETAILED DESCRIPTION

Referring now to FIG. 1, a bioreactor system 10 for growing and separating cells is shown. The system 10 includes a cell culture chamber 15, an agitator 20 and a control system 30.

The cell culture chamber 15 includes an interior 35 for receiving and growing target cells in a cell culture media disposed therein, a first end 40 and a second end 45. As used herein, “target cells” refers to cells disposed within the chamber 15 and which are grown within the chamber 15. While the present disclosure is given the context of growing target cells, it will be appreciated that the system may be employed to mix chemicals or any other suitable solution or material. Also, while the first end 40 and second end 45 are shown as being at the top and bottom of the chamber 15 respectively, it will be appreciated that the ends 40, 45 may be in any suitable orientation relative to one another (e.g., in a horizontal plane) and remain within the scope of the present disclosure. As will be discussed below, the chamber 15 may include one or more interior compartments. In addition, as will be appreciated by those skilled in the art, the chamber 15 may be formed from any suitable material, including a rigid material, a flexible material, a combination of rigid and flexible materials, a gas permeable material or any other suitable material. The chamber 15 may further include one or more ports for providing fluid communication between the chamber interior 35 and one or more reservoirs. Illustrative reservoirs include, without limitation, a cell culture media reservoir, a waste reservoir, a buffer reservoir, a CO₂ reservoir, or any other suitable reservoir.

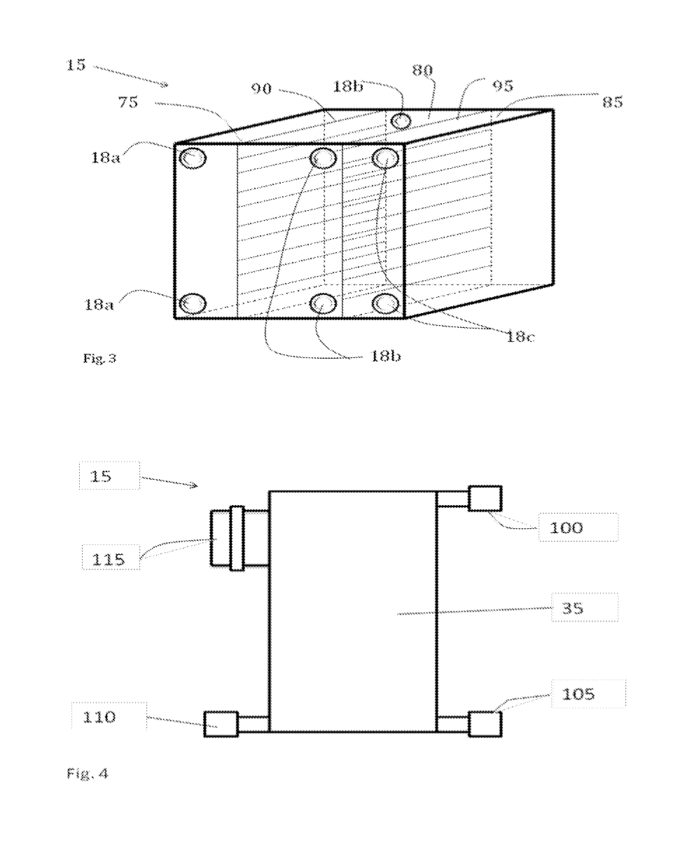

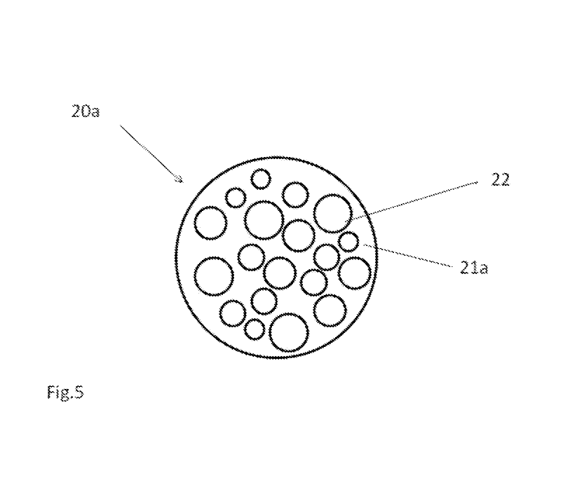

Referring now to FIG. 3, an illustrative alternative chamber 15 is shown. In this embodiment, the chamber 15 includes a first compartment 75, a second compartment 80 and a third compartment 85. The first compartment 75 may include one or more ports 18a for providing fluid communication between the first compartment and one or more reservoirs such as: a cell culture media reservoir, such that media can be added to and/or removed from the first compartment; a buffer reservoir, such that buffer can be added to and/or removed from the first compartment; a waste reservoir, such that waste may be removed from the first compartment 75; a CO₂ reservoir such that CO₂ may be added to and/or removed from the first compartment; or any other suitable reservior. The first compartment 75 and second compartment 80 are separated by a first membrane 90. The first membrane 90 is adapted to selectively permit media to flow from the first compartment 75 to the second compartment 80. The membrane 90 may include perforations, apertures or any other suitable configuration and/or is otherwise semipermeable so as to permit the media to flow from the first compartment 75 to the second compartment 80. In one embodiment, the membrane 90 is a dialysis membrane. The membrane 90 may be formed from any suitable material, including but not limited to cellulose and cellophane.

The second compartment 80 is configured to retain the agitator therein such that the agitator may be moved within the compartment as previously discussed relative to FIG. 1. Target cells are typically grown and retained within the second compartment 80 as well. The second compartment 80 may include one or more ports 18b for providing fluid communication between one or more reservoirs such as the types of reservoirs discussed relative to the first compartment 75. The second compartment 80 and third compartment 85 may be separated by a second membrane 95. The second membrane 95 is adapted to selectively permit media and/or waste to flow from the second compartment 75 to the third compartment 85. The membrane 95 may include perforations, apertures or any other suitable configuration and/or is otherwise semipermeable so as to permit the media and/or waste to flow from the second compartment 80 to the third compartment 85. In one embodiment, the membrane 95 is a dialysis membrane. The membrane 95 may be formed from any suitable material, including but not limited to cellulose and cellophane. The third compartment 85 may include one or more ports 18c for providing fluid communication between the third compartment 85 and one or more reservoirs such as the types of reservoirs discussed relative to the first compartment 75. Also, in at least one embodiment, at least a portion of the exterior of the chamber may be formed from a gas permeable material. When at least a portion of the chamber exterior is formed from a gas permeable material the system may, in some but not necessarily all, instances not include direct gas injection into the chamber and media pH maintaining components. The first compartment 75 and third compartment 85 may be located in any suitable position relative to the second compartment 80 and the present disclosure is not in any way limited to the second compartment 80 positioned between the first and third compartments 75, 80. Additionally, it will be appreciated that any suitable number of compartments may be employed and the present disclosure is in not limited to only three compartments.

In one embodiment, a media comprising a suitable solution having a relatively small number of large and/or large polar molecules is provided to the first compartment 75, and the same or similar media with suitable colloid osmosis for cell growth is provided to the second compartment 80. Suitable media may be any media that used for growing or maintaining the target cells. A buffer having a greater concentration of large and/or large polar molecules to generate and maintain higher colloid osmosis force, as compared to the media of the first and/or second compartments 75, 80, is provided to the third compartment 85. The materials used to generate and maintain higher colloid osmosis force for the third compartment 85 may include, but is not limited to, PEG 80000, albumin, and other proteins or any other suitable material or solution. The membranes 90, 95 may have the same or different permeability and are configured such that media and/or waste from the growth of new cells may flow from the first and/or second chambers 75, 80 to the third chamber 85 via osmosis. The control system may control the infusion and draining of media, buffer and/or waste from one or more compartments 75, 80, 85 so as to maintain a constant volume and osmosis force in each compartment 75, 80, 85 and maintain a supply of fresh media to the second compartment 80. In addition, the control system, via any suitable detection device, mechanism or method, may monitor the any suitable parameter involved in the growth of the target cells, for example and without limitation, the change in the number of target cells, pH, CO₂, glucose, calcium, potassium, sodium, temperature, humidity or any other suitable factor and adjust the interval, frequency and/or speed of the movement of the agitator within the second chamber and/or adjust the amount of media, the type of media, the amount of buffer, the type of buffer, the amount of CO₂, or make any other suitable adjustment based on the control system measurement so as to enhance or promote the growth of the target cells within the chamber.

Referring now to FIG. 4, another illustrative chamber 15 is shown. In this embodiment, the chamber may include one or more ports 100 for providing fluid communication between the chamber interior 35 and a waste reservoir. The chamber 15 may also include one or more ports 110 for providing fluid communication between the chamber interior 35 and a cell culture media reservoir. Also, the chamber 15 may include one or more ports 110 for providing fluid communication between the chamber interior 35 and a buffer reservoir. Further, the chamber may also include one or more ports 115 for receiving the agitator into the chamber interior 35. In one embodiment, the chamber 15 is formed from Teflon FEP. This particular embodiment may be useful in instances where the target cells will adhere or otherwise be coupled to the agitator so that when waste is flushed from the chamber interior 35, the target cells remain within the chamber interior 35. However, it will be appreciated that the chamber 15 may be formed from any suitable material and remain within the scope of the present disclosure.

The agitator 20 is disposed within the chamber interior 35 and is capable of moving between the chamber first end 40 and chamber second end 45. Alternatively, the agitator 20 may be configured to be moved between any two or more points, or between any two or more portions, within the chamber interior 35. In the illustrative embodiment, the agitator comprises a plurality of beads 21. It will be appreciated that any illustrative embodiment showing beads 21 may use any alternative agitator configuration and remain with the scope of the present disclosure and that any particular illustrative embodiment is not limited to using beads exclusively as the agitator. In one embodiment, the beads 21 are be formed from a magnetizable material, such as silicon steel, Fe₃O₄, or any other suitable magnetizable material. As used herein, magnetizable means that the agitator, such as the beads, will hold a magnetic charge when subjected to a magnetic field but will not otherwise hold a magnetic charge once removed from the magnetic field, or the magnetic field removed from the vicinity of the agitator, for example, when a magnetic field generator is de-energized. The magnetizable material typically comprises the core of each bead 21. The magnetizable core may then be coated with any suitable material. In one embodiment, the magnetizable core is coated with polystyrene; however, it will be appreciated that the magnetizable core may be coated with any suitable material and remain within the scope of the present disclosure. For example, and without limitation, the magnetizable core may be coated with any suitable thermoplastic or thermoset polymer. While the beads 21 are shown as being formed from a magnetizable material, it will be appreciated that the beads may be formed from any suitable material, magnetizable or non-magnetizable, and remain within the scope of the present disclosure. Additionally, it will be appreciated that the beads 21 may each be coated with any suitable material such that the target cells will adhere to the beads as the cells grow within the chamber 15, yet it will be appreciated that beads not coated with a particular material to which target cells will adhere also remain within the scope of the present disclosure. In some embodiments, it may be desirable to have beads 21 that are buoyant within the cell culture media; therefore, the core of the beads may include air pockets or bubbles, a lightweight foam or plastic or any other suitable material for permitting the beads 21 to be buoyant within the media.

The beads 21 may be formed such that one or more niches, or micoenvironments, may be formed or created in the voids between the beads 21 when the beads are stacked together. In some embodiments, these niches may promote growth of additional target cells therein. In one embodiment, where the beads are substantially spherical, the diameter of each bead 21 may be between 1 mm and 10 mm for the creation of suitable niches. However, it will be appreciated that the beads 21 may have any suitable size and/or shape such that one or more suitable niches may be formed when the beads 21 are stacked together. Also, it will be appreciated that at least some niches may be formed between some beads and one or more walls of the chamber interior.

In an alternative embodiment, as shown in FIG. 5, the agitator 20a may be a plate member 21a having a plurality of apertures 22 therein. The cross-section of the plate member 21a may be complimentary to the cross-sectional shape of the chamber 15 such that the agitator 20a may move within the chamber interior 35. The apertures 22 may permit the media to flow through the agitator 20a as the agitator 20a moves within the chamber interior 35. The agitator 20a may be formed from magnetizable materials or non-magnetizable material, formed to be buoyant or non-buoyant, and/or coated as previously discussed with respect to the beads 20.

Referring again to FIG. 1, the control system 30 may include one or both of a controller 55 and computer 60 for controlling operation of the system 10. Alternatively, the system 10 may be run manually. The control system 30 is configured to be releasably coupled to the chamber 15. The control system 30 may include a cassette 50 for receiving the chamber 15 but it will be appreciated that the chamber 15 may be coupled to the control system 50 via any suitable means or configuration (e.g., clips, hooks, magnets, hook-and-loop assemblies, friction fit, etc.) and remain within the scope of the present disclosure.

The control system 30 may also include a light source 2 and a cell detector 9 for detecting the number of cells within the chamber 15, detecting the change in the number of cells within the chamber 15 or the like and reporting the results back to the control system 30. However, it will be appreciated that any detector, mechanism or technique known in the art for monitoring the number of cells or the change in the number of cells may be employed and remain within the scope of the present disclosure. Additionally, the control system 30, via any suitable detection device, mechanism or method, may monitor the any suitable parameter involved in the growth of the target cells, for example and without limitation, the change in the number of target cells, pH, CO₂, glucose, calcium, potassium, sodium, temperature, humidity or any other suitable factor and adjust the frequency and/or speed of the movement of the agitator within the chamber and/or adjust the amount of media, the type of media, the amount of buffer, the type of buffer, the amount of CO₂, or make any other suitable adjustment based on any control system measurement so as to enhance or promote the growth of the target cells within the chamber 15.

The control system 30 is operable to cause the agitator to move within the interior 35 of the chamber 15. This may be accomplished a variety of ways. In the illustrative embodiment, the control system includes a motor 65 operable to rotate the chamber 15 between a first position and second position. As will be discussed below, the first position and second position are approximately 180° apart but it will be appreciated that first and second positions may have any suitable angular relationship relative to one another and remain within the scope of the present disclosure. The chamber 15 may be rotated in a horizontal plane, rotated in a vertical plane or rotated, shifted, slid or otherwise moved in any suitable manner to cause the agitator 20 to move within the chamber 15.

In addition, the control system 30 may include first and second magnetic field generators 70, 72 for exciting the beads 21, or other agitator 20, so as to move the beads 21 within the chamber 15 to mix the target cells and culture media. In the illustrative embodiment, each magnetic field generator is an electromagnet that generates a magnetic field when energized and ceases to create a magnetic field when de-energized. When energized, each magnetic field generator draws the agitator 20, e.g. the beads 21, toward the energized magnetic field generator. In an alternative embodiment, a permanent magnet may be used wherein the control system 30 is operable to remove the magnet from the vicinity of the chamber 15 or otherwise block the magnetic field from the magnet from penetrating into the chamber 15. While the illustrative embodiment employs both chamber rotation and electromagnets for moving the agitator within the chamber, it will be appreciated that chamber rotation may be used alone or that electromagnets may be used alone. Moreover, it will be appreciated that any technique for moving the agitator within the chamber may be employed and remain within the scope of the present disclosure.

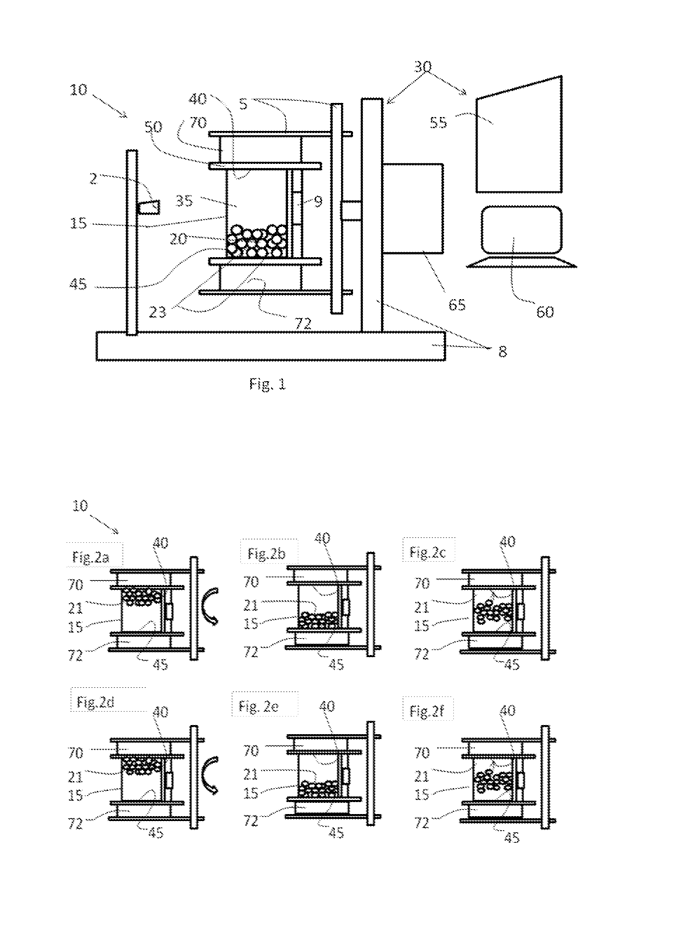

Referring now to FIG. 2a-2f, operation of the system 10 is illustrated by way of a non-limiting example. Target cells and cell culture media are delivered to the interior 35 of the chamber 15. In this embodiment, the beads 21 are buoyant and float near the top of the cell culture media within the chamber 15. In FIG. 2a, the first magnetic field generator 70 is energized and the beads 21 are held near the first end 40 of the chamber 15. The chamber 15 is then rotated approximately 180° to a position as shown in FIG. 2b wherein the first magnetic field generator 70 maintains the beads 21 near the chamber first end 40. The first magnetic field generator 70 may then be de-energized whereby the beads 21 begin to float towards the second end 45 of the chamber 15 as shown in FIG. 2c. In embodiments where the beads 21 include a coating which target cells will adhere to, movement of the beads 21 from one end to the other will collect newly grown target cells. The target cells may adhere to the beads 21 while waste is flushed from the chamber 15 and/or when new media is introduced to the chamber 15 such that a substantial number of the target cells, original and newly grown, remain within the chamber. Alternatively, magnetizable antibodies specific to the target cells may be added to the interior of chamber 15 whereby the antibodies will bind themselves to the target cells, and when a magnetic field is introduced to the chamber, the antibody bound target cells will be releasably coupled to the magnetizable beads 21 and/or the chamber wall(s) adjacent to the magnetic field generator(s). In this embodiment, one or both of the magnetic field generators 70, 72 may remain energized while unbound cells and/or waste are flushed from the chamber and/or while new media is introduced to the chamber such that a substantial number of the target cells, original and newly grown, remain within the chamber 15. Alternatively, magnetic reagents, such as Annexin V or other suitable reagent, may be employed to couple to damaged or dead cells to the beads and the healthy target cells flushed from the system 10. Further, it will be appreciated that magnetizable antibodies and/or reagents may be employed in a chamber 15 without the use of an agitator whereby the target cells or damaged/dead cells may be held against the chamber when the chamber is flushed.

Referring again to FIGS., once the beads 21 are near the second end 45 of the chamber, the second magnetic field generator 72 may be energized whereby the beads 21 are held near the chamber second end 45 (FIG. 2d) and the chamber rotated to the position shown in FIG. 2e. The second magnetic field generator 72 may then be de-energized whereby the beads 21 will float towards the chamber first end 40 as shown in FIG. 2f. As will be appreciated by those skilled in the art, a variety of additives, media, buffers, CO₂ and the like may be selectively added to the chamber at any desired point during this process and/or waste selectively removed in order to promote or enhance new cell growth based on measurements taken by the control system as previously discussed.

In an alternative embodiment, non-buoyant beads may be employed such that the beads are moved within the chamber by rotation of the chamber and without also being subjected to magnetic fields. Here, gravity and centrifugal force, by way of rotation of the chamber, are employed to move the beads between two or more points within the chamber 15. In yet another alternative, the first and second magnetic field generators 70, 72 may be alternately energized so as to move the beads between two or more points within the chamber and without any rotation of the chamber 15. While the forgoing example employs beads 21 as the agitator, it will be appreciated that suitable device may be employed as the agitator and remain within the scope of the present disclosure, including but not limited to that of FIG. 5. Moreover, it will be appreciated that any means or technique for moving the agitator within the chamber may be employed and remain within the scope of the present disclosure.

Moreover, it will be appreciated that if the chamber 15 formed from gas permeable material or otherwise includes a gas permeable portion, the system may be disposed within a CO₂ incubator or CO₂ room. Without a CO₂ incubator or CO₂ room or without any gas permeable portion of the chamber, reagents, such as HEPES may be employed or, alternatively, CO₂ may be injected directly into the chamber from a CO₂ reservoir.

Although the present invention and its advantages have been disclosed in the context of certain illustrative, non-limiting embodiments, it should be understood that various changes, substitutions, permutations, and alterations can be made without departing from the scope of the invention as defined by the appended claims. It will be appreciated that any feature that is described in a connection to any one embodiment may also be applicable to any other embodiment.

The invention claimed is:

1. A bioreactor for the sorting of cells and growth of cells and engineered tissue, which comprises at least one reaction chamber, an adjustable magnetic field, and a multifunctional cell supporting system, which are accompanied with or without a protective perfusion system and/or a computerized control system. Anyone of above five main elements can be used individually or in combination with other elements. Said reaction chamber or chambers are for containing cells, cell-culture media and cell supporters. Said adjustable magnetic field is for controlling cell sorting procedure and culturing procedure. Said multifunctional cell supporting system is for capturing positive cells in cell sorting, supporting adherent cells, forming niches for cell growth and keeping cells in suspension. Said protective perfusion system is for rapid medium change without loss of cells, peptides and proteins. Said computerized control system includes a computer or similar program device and its detectors of reaction conditions as well as its feedback system which controls the bioreactor.

2. The bioreactor system of claim 1 wherein the chamber interior comprises:
a first compartment, the first compartment adapted for fluid communication with a cell culture media reservoir such that the first compartment is capable of receiving cell culture media from the reservoir;
a second compartment, wherein the agitator is disposed within the second compartment;
a first membrane positioned between the first compartment and second compartment, the first membrane adapted to selectively permit cell culture media to flow from the first compartment to the second compartment;
a third compartment, the third compartment adapted for fluid communication with a buffer reservoir such that the third compartment is capable of receiving buffer from the buffer reservoir and wherein the third compartment is further adapted for fluid communication with a waste reservoir such that waste is capable of flowing from the third reservoir to the waste reservoir;
a second membrane positioned between the second compartment and third compartment, the second membrane adapted to selectively permit flow of media and waste from the second compartment to the third compartment; and at least one side wall of the reaction chamber made with gas-permeable materials, otherwise gas perfusion system or/and certain media components, such as HEPES, should be applied. The chamber can be fixed to the flipping arm of the bioreactor by a cassette and any holders.

3. The bioreactor of claim 1 wherein said multifunctional cell supporting system consists of magnetizable agitators which are either naked (uncoated) or coated with inert or any other materials, for any purpose including attachment of cells and reducing cell damage. The bioreactor system of claim 1 wherein the agitator comprises plurality of beads or plate member having a plurality apertures therein. Said magnetizable agitators at least have one part made with magnetizable material which becomes magnetic when placed inside a magnetic field but is not magnetic or very weakly magnetic when the magnetic field disappears or is removed from the reaction area.
The size of said beads is greater than 1 millimeter in diameter and smaller than the volume of the said reaction chamber in claim 1 and claim 2.

4. The bioreactor of claim 3 wherein said magnetizable agitators serve as cell supporter for the cell sorting and adherent cell culture. In the cell sorting, they are used to catch magnet-labeled cells in the cell sorting (cell selection, isolation or purification as said and in some situations), to allow the cells to adhere to the surface of the said magnetizable agitators. The magnetic labeling is usually mediated by antibodies but may be achieved via other means. When the magnetic field disappears or is removed, the magnetically-labeled cells are released from the beads. In the adherent cell culture, magnetizable agitators allow the cells to attach them and grow on them.

5. The bioreactor of claim 3 wherein said magnetizable agitators are used to gently agitate media by moving between any two ends within the reaction chamber to keep cells suspended in the liquid growth medium or to gently agitate media chamber to keep cells suspended. The magnetizable agitators can move in the bioreactor via but not limited to the following three mechanisms: 1) attraction to the surrounding magnetic field and 2) buoyancy, when the beads are less dense than the medium in the reaction chamber, or 3) gravity, when the beads are denser than the medium in the reaction chamber.

6. The bioreactor of claim 3 wherein said magnetizable agitators are used to create a “niche” or microenvironment for cell growth. As the magnetizable agitators settle down after each movement (by the mechanisms mentioned in claim 5), the spaces between the beads create a perfect niche for cell growth, with prevent any overcrowding or clumping that may impair cell growth and reduce stem cell non-specific differentiation.

7. The bioreactor of claims 1, 3, 4, 5 and 6 wherein said magnetic field changes can be the changes of magnetic field strength and/or direction, but its position relative to the reaction chamber in the bioreactor can be fixed or not.

8. The bioreactor of claim 1 wherein said adjustable magnetic field can be generated by an electric magnet which adjusts the magnetic field by changing its electric current and/or voltage or by changing the position and direction (orientation) of the electric magnet. Said adjustable magnetic field can also be generated via a permanent magnet that alters the magnetic field by changing the position and direction of the permanent magnet.

9. The bioreactor of claim 1 wherein said protective perfusion system comprises one or more dialysis membrane walls between the reaction compartment and gradient osmosis compartments of the chamber, one or more gradient osmosis compartments adjacent to reaction compartments, gradient osmosis buffers, gradient osmosis buffer reservoirs, relevant dynamic devices for osmosis buffer circulating and a waste collection container. The fresh medium from the lower osmosis compartment passes through the dialysis membrane into the reaction compartment (cell culture compartment) while the old medium from reaction compartment pass through the dialysis membrane into the high osmosis compartment before being discharged into a waste container.

10. The bioreactor of claim 9 wherein said gradient osmosis buffers comprises a high osmosis buffer that contains a high concentration of large molecules, such as proteins and PEG 8000 to form high colloid osmotic force, as well as a low osmosis buffer that contains little to no large molecules.

11. The bioreactor of claim 1 wherein said computerized control system includes a computer attached to detectors of reaction conditions and a feedback system. Conditions monitored by the detectors include but are not limited to: cell density (concentration), pH, concentrations of glucose, CO₂, O₂, N₂, as well as temperature and humidity in the reaction chamber. The said detector can be a sensor or a combined device with a sensor and signal generator. These sensors can directly input data into an internal or external computer. After analyzing the data, the computer gives the bioreactor a feedback to adjust those factors to meet the requirements of cell growth. The computer also offers pre-selected programs that operate the bioreactor. The control system also includes a motor or a servo, wherein the motor (or servo) is coupled to the cell culture chamber and operable to rotate the chamber between a first position and a second position, wherein rotation of the chamber from the first position to the second position causes the agitator to move from the chamber interior first portion towards the chamber interior second portion.

12. Methods for sorting cells and growing cells and engineered tissue include: preparing cells for sorting, isolating the target cells in the bioreactor, discharging unselected cells and old medium, real time monitoring of cell density and cell growth condition, real time adjusting and optimizing the cell culture conditions for the cell growth and directed differentiation, programming and applying the computerized system for adjusting and optimizing cell culture condition, culturing the cells in the culture chamber with or without niches and collecting the cells in the bioreactor said in claim 1.

13. The methods of claim 12 wherein said sorting cells and growing cells are conducted in the same reaction chamber. The cell sorting can be done before, after and during cell culture.

14. The methods of claim 12 wherein said cell sorting can be done with magnetically-labeled cells caught by magnetizable agitators or/and chamber walls close to the magnetic field generator (magnet or electric magnet) in claim 3, in which magnetizable agitators are controlled by adjustable magnetic field in claim 1. Any non-targeted cells can be washed out and discharged. Target cells can be immediately released from beads when the magnetic field is removed, such as switching off the electric magnet.

15. The methods of claim 14 wherein said magnetically-labeled cells can be prepared with magnetically-labeled antibody, other materials, or unlabeled.

16. The methods of claim 12 wherein said real time monitoring cell density can be conducted with the principles of photology or/and biochemistry.

17. The methods of claim 12 wherein growing cells is conducted when the bioreactor keeps cells in suspension status or in static status or in a program that alternates between suspended and static states.

18. The methods of claim 17 wherein said cells may be kept in suspension via the movement of magnetizable agitators, which is controlled by an adjustable magnetic field with or without the help of gravity and/or buoyancy.

19. The methods of claim 18 wherein said adjustable magnetic field-controlled moving magnetizable agitators can move between any two ends of the reaction chamber in all directions, which is controlled by adjustable magnetic field with or without the help of gravity and/or buoyancy, though movement along the vertical axis is preferred.

20. The methods of claim 12 wherein said niches form among the magnetizable agitators when the magnetizable agitators are physically still. These niches can also be adjusted by moving magnetizable agitators.

21. The methods of claim 12 wherein said computerized control system is programmable and programmed for all or part of the bioreactor's functions.

22. The methods of claim 12 wherein said real time adjusting and optimizing the cell culture conditions for the cell growth and directed differentiation are carried out by a computer controlled combined system including cell culture condition detectors, computer or similar devices and corresponding programs, and feedback operation control system.

23. The methods of claim 12 wherein said the cell culture conditions for directed differentiation are for all cells which have differentiation potential, including but not limited to all kinds of stem cells. The said directed differentiation can be induced simultaneously by biochemical and physical methods for two or more directions of induction, such as inducing hepatocytes and their blood vessels at the same time.

24. The bioreactor of claim 1 wherein said current invented bioreactor can provide ideal cell growth conditions for all cells, including but not limited to hematopoietic stem cells, mesenchymal stem cells, Fibroblasts, hybridoma cells, lymphocytes, dendritic cells, insect cells, embryonic stem cells, various tissue cells, cancer cells and cell lines, and transformed cells and cell lines.

25. The bioreactor of claim 1 wherein said all components and related methods are not only used in cell culture, but also in any field that benefit from this system and methods.
